(19) United States
(12) Patent Application Publication
(10) Pub. No.: US 2003/0215816 A1
(43) Pub. Date: Nov. 20, 2003

(54) Method for sequencing nucleic acids by observing the uptake of nucleotides modified with bulky groups

(76) Inventors: Narayan Sundararajan, San Francisco, CA (US); Andrew A. Berlin, San Jose, CA (US); Mineo Yamakawa, Campbell, CA (US); Xing Su, Cupertino, CA (US); Selena Chan, Sunnyvale, CA (US); Tae-Woong Koo, South San Francisco, CA (US)

Correspondence Address: Blakely, Sokoloff, Taylor & Zafman, Seventh Floor, 12400 Wilshire Boulevard, Los Angeles, CA, 90025-1030, US

(21) Appl. No.: 10/153,189

(22) Filed: May 20, 2002

(51) Int. Cl. C12Q 1/68; C12P 19/34

(52) U.S. Cl. 435/6; 435/91.2

(57) ABSTRACT

The present methods and apparatus 100 concern nucleic acid 214 sequencing by incorporation of nucleotides 218 into nucleic acid strands 220. The incorporation of nucleotides 218 is detected by changes in the mass and/or surface stress of the structure 116, 212. In some embodiments of the invention, the structure 116, 212 comprises one or more nanoscale or microscale cantilevers. In certain embodiments of the invention, each different type of nucleotide 218 is distinguishably labeled with a bulky group and each incorporated nucleotide 218 is identified by the changes in mass and/or surface stress of the structure 116, 212 upon incorporation of the nucleotide 218. In alternative embodiments of the invention only one type of nucleotide 218 is exposed at a time to the nucleic acids 214, 220. Changes in the properties of the structure 116, 212 may be detected by a variety of methods, such as piezoelectric detection, shifts in resonant frequency of the structure 116, 212, and/or position sensitive photodetection.

### BACKGROUND OF THE INVENTION

1. Field of the Invention

The methods and apparatus described herein relate to the fields of molecular biology and nucleic acid analysis. In particular, the disclosed methods and apparatus relate to sequencing nucleic acids by detecting changes in mass and/or surface stress upon incorporation of labeled nucleotides.

2. Background

Genetic information is stored in the form of very long molecules of deoxyribonucleic acid (DNA), organized into chromosomes. The human genome contains approximately three billion bases of DNA sequence. This DNA sequence information determines multiple characteristics of each individual. Many common diseases are based at least in part on variations in DNA sequence.

Determination of the entire sequence of the human genome has provided a foundation for identifying the genetic basis of such diseases. However, a great deal of work remains to be done to identify the genetic variations associated with each disease. That would require DNA sequencing of portions of chromosomes in individuals or families exhibiting each such disease, in order to identify specific changes in DNA sequence that promote the disease. Ribonucleic acid (RNA), an intermediary molecule in processing genetic information, may also be sequenced to identify the genetic bases of various diseases.

Existing methods for nucleic acid sequencing, based on detection of fluorescently labeled nucleic acids that have been separated by size, are limited by the length of the nucleic acid that can be sequenced. Typically, only 500 to 1,000 bases of nucleic acid sequence can be determined at one time. This is much shorter than the length of the functional unit of DNA, referred to as a gene, which can be tens or even hundreds of thousands of bases in length. Using current methods, determination of a complete gene sequence requires that many copies of the gene be produced, cut into overlapping fragments and sequenced, after which the overlapping DNA sequences may be assembled into the complete gene. This process is laborious, expensive, inefficient and time-consuming. It also typically requires the use of fluorescent or radioactive labels, which can potentially pose safety and waste disposal problems.

More recently, methods for nucleic acid sequencing have been developed involving hybridization to short oligonucleotides of defined sequenced, attached to specific locations on DNA chips. Such methods may be used to infer short nucleic acid sequences or to detect the presence of a specific nucleic acid in a sample, but are not suited for identifying long nucleic acid sequences.

### BRIEF DESCRIPTION OF THE DRAWINGS

The following drawings form part of the specification and are included to further demonstrate certain embodiments of the invention. The embodiments may be better understood by reference to one or more of these drawings in combination with the detailed description presented herein.

FIG. 1 illustrates an exemplary apparatus 100 (not to scale) for nucleic acid 214 analysis.

FIG. 2A, FIG. 2B and FIG. 2C illustrate another exemplary embodiment of an apparatus 100 (not to scale) for nucleic acid 214 analysis.

FIG. 3 illustrates an example of sequencing data that may be generated using the methods and apparatus 100 described herein.

FIG. 4 illustrates another example of sequencing data that may be generated using the methods and apparatus 100 described herein.

### DESCRIPTION OF ILLUSTRATIVE EMBODIMENTS

Definitions

As used herein, “a” and “an” may mean one or more than one of an item.

As used herein, “about” means within plus or minus five percent of a number. For example, “about 100” means any number between 95 and 105.

As used herein, “operably coupled” means that there is a functional interaction between two or more units. For example, a detection unit 118 may be “operably coupled” to a structure 116, 212 if the detection unit 118 is arranged so that it may detect changes in the properties of the structure 116, 212.

As used herein, “fluid communication” refers to a functional connection between two or more compartments that allows fluids to pass between the compartments. For example, a first compartment is in “fluid communication” with a second compartment if fluid may pass from the first compartment to the second and/or from the second compartment to the first compartment.

“Nucleic acid” 214 encompasses DNA, RNA, single-stranded, double-stranded or triple stranded and any chemical modifications thereof. In certain embodiments of the invention single-stranded nucleic acids 214 may be used. Virtually any modification of the nucleic acid 214 is contemplated. A “nucleic acid” 214 may be of almost any length, from 10, 20, 50, 100, 200, 300, 500, 750, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100,000, 150,000, 200,000, 500,000, 1,000,000, 2,000,000, 5,000,000 or even more bases in length, up to a full-length chromosomal DNA molecule.

### DETAILED DESCRIPTION OF ILLUSTRATIVE EMBODIMENTS

The methods and apparatus 100 disclosed herein are of use for the rapid, automated sequencing of nucleic acids 214. Advantages over prior art methods include the ability to read long nucleic acid 214 sequences in a single sequencing run, greater speed of obtaining sequence data, decreased cost of sequencing and greater efficiency in operator time required per unit of sequence data. In some embodiments of the invention, the ability to sequence nucleic acids 214 without using fluorescent or radioactive labels is also advantageous.

The following detailed description contains numerous specific details in order to provide a more thorough understanding of the disclosed embodiments of the invention. However, it will be apparent to those skilled in the art that the embodiments of the invention may be practiced without these specific details. In other instances, devices, methods, procedures, and individual components that are well known in the art have not been described in detail herein.

Certain embodiments of the invention concern methods and apparatus 100 for nucleic acid 214 sequencing. In some embodiments of the invention, nucleic acids 214 to be sequenced may be attached to one or more structures 116, 212, such as nanoscale or microscale cantilevers 116, 212. In various embodiments of the invention, the attached nucleic acids 214 may serve as templates for production of complementary strands 220 or for the replication of duplicate nucleic acids 214. In some embodiments of the invention, the nucleotides 218 used for synthesis of complementary strands 220 may be tagged with bulky groups, providing a unique mass label for each type of nucleotide 218. The nucleic acids 214, 220 may be incubated in a solution containing all four types of labeled nucleotides 218. As each nucleotide 218 is added to a growing strand 220, it adds to the mass attached to the structure 116, 212. Because each nucleotide 218 may be identified by its unique mass, it is possible to identify the nucleotides 218 in their order of addition by measuring mass-dependent properties and/or changes in surface stress of the structures 116, 212, such as their resonant frequency or deflection. It is contemplated in various embodiments of the invention that multiple copies of the same nucleic acid template 214 may be attached to each structure 116, 212 and that synthesis of many complementary strands 220 may occur simultaneously, providing a sufficient increase in mass and/or change in surface stress to be detectable upon addition of each nucleotide 218 in sequence.

In alternative embodiments of the invention, the growing complementary nucleic acids 220 may be exposed to only a single type of nucleotide 218 at one time. Incorporation of nucleotides 218 would only occur when the nucleotide 218 is complementary to the corresponding nucleotide 218 in the template strand 214. Thus, the mass of nucleic acids 214, 220 attached to the structure 116, 212 and/or surface stress of the structure will only change when the correct nucleotide 218 is present. The addition of consecutive nucleotides 218 of identical type is indicated by a correspondingly larger change in the mass and/or surface stress. In such embodiments, it is not necessary that each type of nucleotide 218 have a distinguishable mass label.

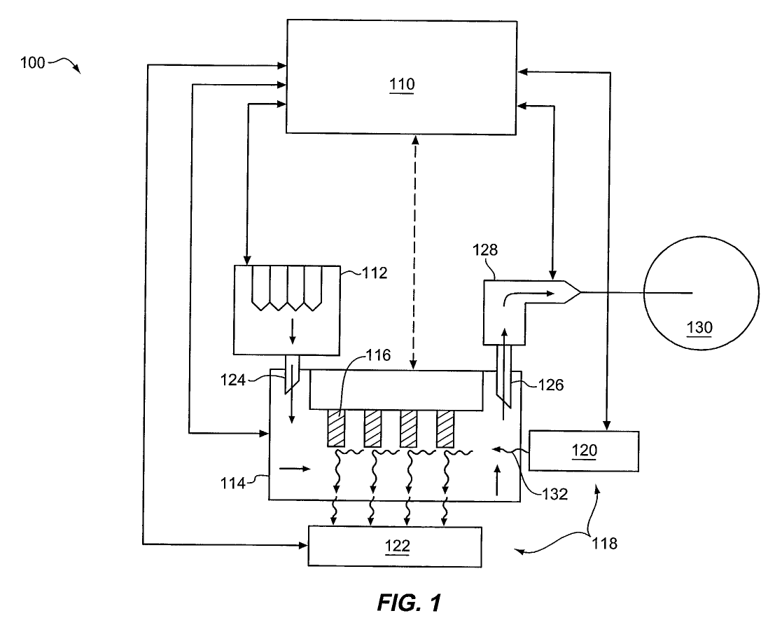

Various embodiments of the invention concerning an exemplary apparatus 100 for nucleic acid 214 sequencing are illustrated in FIG. 1. The apparatus 100 of FIG. 1 comprises a data processing and control unit 110 that is operably coupled to other components of the apparatus 100, such as a reagent reservoir 112, an analysis chamber 114, 210 a detection unit 118, and outlet 128. The reagent reservoir 112 of FIG. 1 is in fluid communication with an analysis chamber 114, 210 via an inlet 124. The analysis chamber 114, 210 includes one or more structures 116, 212 for attaching template nucleic acids 214. A microfluidic device may be incorporated to transport enzymes, labeled nucleotides 218, and/or other reagents to and from the analysis chamber 114, 210.

Nucleic acid strands 220 complementary in sequence to the template nucleic acid 214 may be synthesized by known techniques, for example using any of the known nucleic acid polymerases 222. Incorporation of labeled nucleotides 218 into the complementary strands 220 may be detected by measuring any mass dependent property and/or the surface stress of the attached structure 116, 222.

Non-limiting examples of structures 116, 212 that may used include a cantilever, a diaphragm, a platform suspended or supported by springs or other flexible structures, or any other structure 116, 212 known in the art for which measurement of mass dependent properties and/or surface stress, such as deflection and/or resonant frequency shifts may be performed. An example of an appropriate structure 116, 212 is a cantilever 116, 212, as shown in FIG. 1. Known microfabrication techniques may be use to fabricate an analysis chamber 114, 210 with one or more such structures 116, 212 (e.g., Bailer et al., 2000, Ultramicroscopy. 82:1-9; U.S. Pat. No. 6,073,484). Techniques for fabrication of nanoscale cantilever 116, 212 arrays are known. (E.g., Baller et al., 2000; Lang et al., Appl. Phys. Lett. 72:383, 1998; Lang et al., Analytica Chimica Acta 393:59, 1999; see also http://monet.physik.unibas.ch/nose/inficon/; http://www.phantomsnet.com/phantom/net/phantomsconf/doc/Abadal.pdf; http://lmn.web.psi.ch/annrep/mntech3.pdf; www.nnf.cornell.edu/2001cnfra/200138.pdf; http://www.princeton.edu/˜cml/html/research/biosensor.html) In alternative embodiments of the invention, piezoelectric materials such as quartz crystal microbalances may be used as structures 116, 212. (E.g., Zhou et al., Biosensors & Bioelectronics 16:85-95, 2001; Yamaguchi et al., Anal. Chem. 65:1925-1927; Bardea et al., Chem. Commun. 7:839-40, 1998.)

One or more template nucleic acids 214 may be attached to each cantilever 116, 212. A detection unit 118 monitors the position and/or resonant frequency of the cantilevers 116, 212. In some embodiments of the invention, the detection unit 118 may comprise a light source 120, operably coupled to a photodetector 122. Alternatively, a piezoelectric sensor may be operably coupled to a detector 122 or directly coupled to a data processing and control unit 110.

The exemplary embodiment of the invention illustrated in FIG. 1 shows optical detection of the deflection of a cantilever 116, 212. The detection method is based on an optical lever technique, as known for atomic force microscopy (AFM). A low power laser beam 132 may be focused onto the free end of a cantilever 116, 212. The reflected laser beam 132 strikes a position sensitive photodetector 122 (PSD). When the cantilever 116, 212 bends in response to a change in the mass of attached nucleic acids 214, 220 and/or the surface stress of the cantilever 116, 212, the position that the reflected laser beam 132 strikes the PSD 122 moves, generating a deflection signal. The change in mass and/or surface stress and consequent degree of deflection of the cantilever 116, 212 may be calculated from the displacement of the reflected laser beam 132 on the PSD 122.

In various embodiments of the invention, solutions of labeled nucleotides 218 may be introduced into the analysis chamber 114, 210 one labeled nucleotide 218 at a time. For example, a solution comprising a labeled guanine (“G”) nucleotide 218 may be introduced into the analysis chamber 114, 210 via a reagent reservoir 112. The solution may be incubated for an appropriate amount of time with template nucleic acid 214, a primer 224 or complementary nucleic acid 220 and polymerase 222. If the next nucleotide 218 in the sequence of the template nucleic acid 214 is a cytosine (“C”), then a labeled G will be incorporated into the growing complementary nucleic acid 220 strand and a corresponding change in the structure detected. If the next nucleotide 218 of the template nucleic acid 214 is not a C then no change will be detected. The solution containing labeled G nucleotide 218 is removed from the analysis chamber 114, 210 and a solution containing the next labeled nucleotide 218 (adenine—“A”, thymine—“T” or cytosine—“C” is introduced. After all four labeled nucleotide 218 solutions have been cycled through the analysis chamber 114, 210, the cycle repeats itself and continues until the nucleic acid 214 has been sequenced. The sequence of the template nucleic acid 214 may be determined by correlating the measured changes in the properties of the structure with the order in which different nucleotides 218 are exposed to the template 214. Where multiple nucleotides 218 of the same type are incorporated into the complementary strand 220, a proportional change in the properties of the structure 116, 212 will be noted. For example, if incorporation of a single nucleotide 218 produces a change of “X” in a property of the structure 116, 212, then the incorporation of two or three nucleotides of the same type would be expected to result in changes of about 2X or 3X, respectively.

In alternative embodiments of the invention, part of the sequence of the target nucleic acid 214 may be known. For example, the nucleic acid 214 may have already been partially sequenced, or an unknown nucleic acid 214 sequence may have been ligated to vector, linker or other DNA of known sequence. In this case, rather than cycling through all four nucleotides 218, the correct nucleotide 218 for the next addition in sequence may be added until an unknown sequence region is reached. Use of partial known sequences may also serve to calibrate the system and check for proper function. In certain embodiments, for example where a single nucleotide polymorphism (SNP) is to be analyzed, the entire nucleic acid 214 sequence may be known except for a single position, which typically will contain one of two nucleotides 218. Such embodiments allow for even more efficient cycling of nucleotides 218 through the analysis chamber 114, 210.

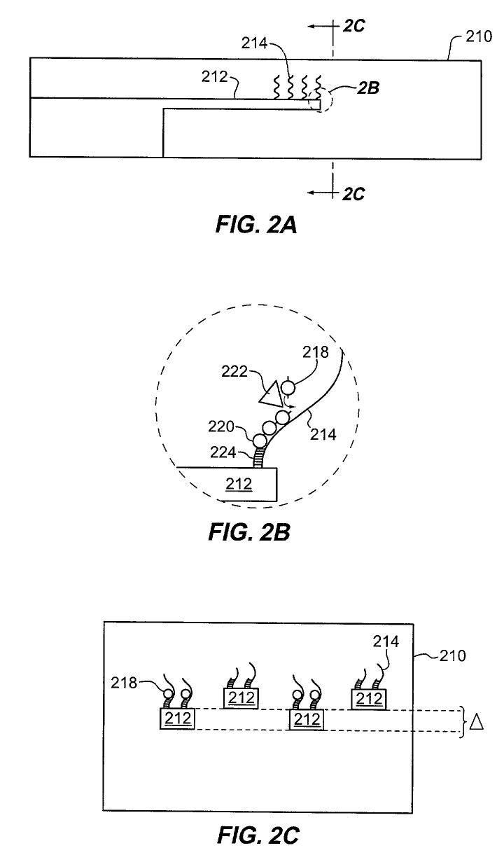

FIG. 2A, FIG. 2B and FIG. 2C illustrate detailed views of an exemplary analysis chamber 114, 210, including a cantilever 116, 212, and template nucleic acids 214 attached to the cantilever 116, 212. FIG. 2B illustrates an expanded view of a single template nucleic acid 214 attached to the cantilever 116, 212. The template 214 hybridizes with a primer 224 oligonucleotide that is complementary in sequence to the 3′ end of the template molecule 214. A nucleic acid polymerase 222, such as a DNA polymerase 222, attaches to the 3′ end of the primer 224 and begins to synthesize a complementary strand 220. Each nucleotide 218 in sequence is added to the 3′ end of the primer 224 or the complementary strand 220 by the polymerase 222. The sequence of the complementary strand 220 is determined by standard Watson-Crick base-pair formation with the template strand 214, where A only binds with T (or uracil—“U” in the case of an RNA template 214) and C only binds with G. Although the embodiment of the invention discussed herein contemplates synthesis of a complementary strand 220 of DNA from a DNA template strand 214, it is contemplated in alternative embodiments of the invention that an RNA template 214 could be used for synthesis of a complementary RNA or DNA strand 220, or that a DNA template 214 may be used for synthesis of a complementary RNA strand 220. In the case of RNA synthesis, for example using an RNA polymerase 222, no primer 224 would be required.

Changes in mass and/or surface stress upon incorporation of nucleotides 218 may be detected by deflection or resonant frequency shift of the cantilever 116, 212 using optical detection methods or piezoelectric devices (see U.S. Pat. Nos. 6,079,255 and 6,033,852). FIG. 2C illustrates an exemplary method of detecting the deflection (Ad) of a cantilever 116, 212 in response to nucleotide 218 incorporation. To increase accuracy and decrease background noise, the position of the cantilevers 212 containing newly incorporated nucleotides 218 may be compared to the position of one or more control cantilevers 212 in which nucleotide 218 incorporation has been blocked, for example by use of a dideoxynucleotide at the 3′ end of the primer 224. As is known in the art, dideoxynucleotides act to block or terminate nucleic acid 220 synthesis.

In various alternative embodiments of the invention, nucleotides 218 may be uniquely labeled with a bulky group, such as nanoparticles and/or nanoparticle aggregates of distinct mass, which may be used to identify each type of nucleotide 218. Solutions of nucleotides 218 may contain one, two, three, or four different types of labeled nucleotides 218 (A, G, C and T or U). In certain alternative embodiments of the invention, only two out of four types of nucleotides 218 may be mass labeled, for example, A and C nucleotides 218. The difference in mass between unlabeled pyrimidine (C, T or U) and purine (A, G) nucleotides 218 should be distinguishable by mass and/or surface stress detection, as should the difference between labeled and unlabeled nucleotides 218.

The identity of the nucleotide 218 incorporated into a complementary nucleic acid 220 strand may be determined by distinctive changes in mass and/or surface stress and the order in which the changes occur. In certain embodiments of the invention, each nucleotide 218 may be labeled with a unique bulky group. The identity of an incorporated labeled nucleotide 218 may be determined from the distinctive change in mass and/or surface stress of the structure 116, 212. In alternative embodiments of the invention each nucleotide 218 may be labeled with the same or a similar bulky group. By identifying the sequence of addition of labeled nucleotides 218 to elongating complementary nucleic acid strands 220, the sequence of the template nucleic acid strand 214 may be determined.

In certain embodiments of the invention, the nucleotides 218 to be added may be DNA precursors—deoxyadenosine 5′ triphosphate (dATP) 218, deoxythymidine 5′ triphosphate (dTTP) 218, deoxyguanosine 5′ triphosphate (dGTP) 218 and deoxycytosine 5′ triphosphate (dCTP) 218. In alternative embodiments of the invention, the nucleotides 218 may be RNA precursors such as adenosine 5′ triphosphate (ATP) 218, thymidine 5′ triphosphate (TTP) 218, guanosine 5′ triphosphate (GTP) 218 and cytosine 5′ triphosphate (CTP) 218

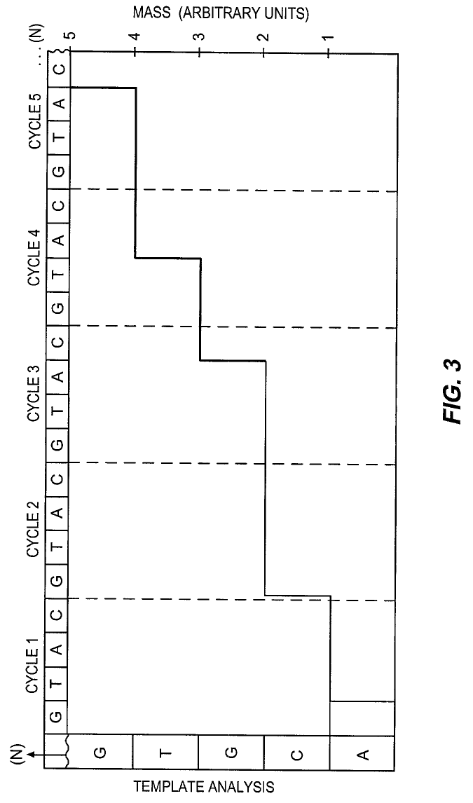

An illustration of exemplary data that may be obtained using sequential exposure to single nucleotide 218 solutions is provided in FIG. 3. As indicated, for each cycle the template 214, primer 224 or complementary strand 220, and polymerase 222 will be sequentially exposed to each of the four nucleotide 218 types (G, T, A and C). In cycle 1, a change in mass and/or surface stress is observed when the T solution is added, indicating the presence of a corresponding A on the template 214. In cycle 2, a change in mass and/or surface stress is seen when the G solution is added, indicating a C in the template 214, etc. The linear sequence of the template 214 may be identified by continuing the cyclic additions and measurements.

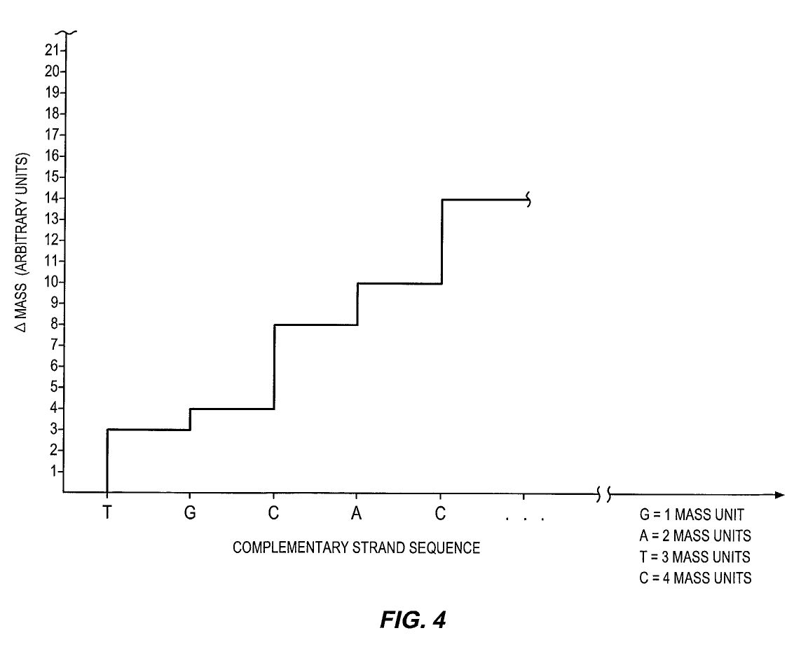

An example of data that may be obtained using an alternative method wherein all four nucleotides 218 are distinguishably labeled and added in the same solution is illustrated in FIG. 4. The mass labels are arbitrarily selected for purposes of illustration such that G has a single mass unit, A has 2 mass units, T has 3 mass units and C has 4 mass units. The skilled artisan will realize that the precise values of the mass units are unimportant, so long as they are distinguishable for each of the four types of nucleotides 218. As shown in FIG. 4, the first nucleotide 218 added has a mass of 3 units, corresponding to T, the second nucleotide 218 added has a mass of 1 unit, corresponding to a G, the third nucleotide 218 has a mass of 4 units, corresponding to C, etc. Reading the complementary 220 sequence from 5′ to 3′, the sequence shown is TGCAC. The corresponding sequence of the template 214 strand, from 3′ to 5′ would be ACGTG.

In embodiments of the invention involving multiple template strands 214 exposed to mixtures of all four nucleotides 218, the polymerization reaction may be synchronized, for example by controlled changes in temperature, adding aliquots of polymerase 222 and/or primers 224 with rapid mixing, or similar known techniques so that the same nucleotide 218 is added to each complementary strand 220 simultaneously. For longer sequencing runs, periodic resynchronization of the polymerization reactions may be required. Alternatively, synchronized polymerization may utilize one or more protecting groups at the 3′ terminus of the complementary nucleic acid strands 220. Additional nucleotides 218 may be incorporated only after removing the protecting group of a previously incorporated nucleotide 218. The addition and cleavage of protecting groups from nucleotides 218 are well known and may include chemically and/or photocleavable groups, as discussed in U.S. Pat. No. 6,310,189.

In embodiments of the invention where labeled nucleotides 218 are used, long template strands 214 may be sequenced in stages to avoid or reduce the possible effects of steric hindrance from the bulky groups used for labeling. Steric hindrance may potentially interfere with the activity of nucleic acid polymerases 222. In a non-limiting example, to sequence a template DNA molecule 214, a primer 224 may be added and the first ten bases sequenced by adding solutions containing single labeled nucleotides 218 (A, G, T or C), as discussed above. After synthesis, the labeled nucleotides 218 may be removed, for example using exonuclease activity, and replaced with unlabeled nucleotides 218 by exposure to solutions containing single unlabeled nucleotides 218. The next ten bases in the template 214 may be sequenced by exposure to solutions containing single labeled nucleotides 218, then the labeled nucleotides 218 replaced with unlabeled nucleotides 218. The process may be repeated until the entire template 214 is sequenced. The skilled artisan will realize that this illustration is exemplary only and that the method is not limited to sequencing ten bases at a time. It is well within the skill in the art to determine the number of contiguous labeled nucleotides 218 that may be incorporated into a complementary strand 220 before substantial interference with polymerase 222 activity occurs. That number may depend in part on the type of polymerase 222 and the types of labels used.

In certain embodiments of the invention the quantity of template nucleic acid molecules 214 bound to a cantilever 116, 212 may be limited. In other embodiments of the invention, template nucleic acids 214 may be attached to one or more cantilevers 116, 212 in particular patterns and/or orientations to obtain an optimized signal. The patterning of the template molecules 214 may be achieved, for example, by coating the structure 116, 212 with various known functional groups, as discussed below.

The analysis of template nucleic acids 214 may provide information about a biological agent or a disease state in a timely and cost effective manner. The information obtained from analysis of nucleic acids 214 may be used to determine effective treatments, such as vaccine administration, antibiotic therapy, anti-viral administration or other treatment.

Micro-Electro-Mechanical Systems (MEMS)

Micro-Electro-Mechanical Systems (MEMS) are integrated systems comprising mechanical elements, sensors, actuators, and electronics. All of those components may be manufactured by known microfabrication techniques on a common chip, comprising a silicon-based or equivalent substrate (e.g., Voldman et al., Ann. Rev. Biomed. Eng. 1:401425, 1999). The sensor components of MEMS may be used to measure mechanical, thermal, biological, chemical, optical and/or magnetic phenomena. The electronics may process the information from the sensors and control actuator components such pumps, valves, heaters, coolers, filters, etc. thereby controlling the function of the MEMS.

The electronic components of MEMS may be fabricated using integrated circuit (IC) processes (e.g., CMOS, Bipolar, or BICMOS processes). They may be patterned using photolithographic and etching methods known for computer chip manufacture. The micromechanical components may be fabricated using compatible “micromachining” processes that selectively etch away parts of the silicon wafer or add new structural layers to form the mechanical and/or electromechanical components. Basic techniques in MEMS manufacture include depositing thin films of material on a substrate, applying a patterned mask on top of the films by photolithograpic imaging or other known lithographic methods, and selectively etching the films. A thin film may have a thickness in the range of a few nanometers to 100 micrometers Deposition techniques of use may include chemical procedures such as chemical vapor deposition (CVD), electrodeposition, epitaxy and thermal oxidation and physical procedures like physical vapor deposition (PVD) and casting.

The manufacturing method is not limiting and any methods known in the art may be used, such as laser ablation, injection molding, molecular beam epitaxy, dip-pen nanolithograpy, reactive-ion beam etching, chemically assisted ion beam etching, microwave assisted plasma etching, focused ion beam milling, electron beam or focused ion beam technology or imprinting techniques. Methods for manufacture of nanoelectromechanical systems may be used for certain embodiments of the invention. (See, e.g., Craighead, Science 290:1532-36, 2000.) Various forms of microfabricated chips are commercially available from, e.g., Caliper Technologies Inc. (Mountain View, Calif.) and ACLARA BioSciences Inc. (Mountain View, Calif.).

In various embodiments of the invention, it is contemplated that some or all of the components of the nucleic acid sequencing apparatus 100 exemplified in FIG. 1 and FIG. 2 may be constructed as part of an integrated MEMS device

Cantilevers

In certain embodiments of the invention, the structure 116, 212 to which the nucleic acids 214, 220 are attached comprises one or more cantilevers 116, 212. A cantilever 116, 212 is a small, thin elastic lever that is attached at one end and free at the other end. Methods of fabricating cantilever 116, 212 arrays are known (e.g., Baller et al., Ultramicroscopy 82:1-9, 2000; U.S. Pat. No. 6,079,255). Cantilevers 116, 212 used for atomic force microscopes are typically about 100 to 200 micrometers (μm) long and about 1 μm thick. Silicon dioxide cantilevers 116, 212 varying from 15 to 400 μm in length, 5 to 50 μm in width and 320 nanometers (nm) in thickness that were capable of detecting binding of single E. coli cells have been manufactured by known methods (Ilic et al., Appl. Phys. Lett. 77: 450, 2000). The material is not limiting, and any other material known for cantilever 116, 212 construction, such as silicon or silicon nitride may be used. In other embodiments of the invention, cantilevers 116, 212 of about 50 μm length, 10 μm width and 100 nm thickness may be used. In certain embodiments of the invention, nanoscale cantilevers 116, 212 of even smaller size may be used, as small as 100 nm in length. In some embodiments of the invention, cantilevers 116, 212 of between about 10 to 500 μm in length, 1 to 100 μm in width and 100 nm to 1 μm in thickness may be used.

When a cantilever 116, 212 is induced to resonate, it can deflect a laser beam 132 focused on the free end of the cantilever 116, 212. By measuring the cantilever 116, 212 deflections with a light detector 122, the resonant oscillation frequency of the cantilever 116, 212 may be determined. Alternatively, deflection of a cantilever 116, 212 may be determined by using a position sensitive photodetector 122 to measure the position of reflected light beams 132 and thereby determine the position of the cantilever 116, 212. These methods are not limiting and any known method for measuring changes in the properties of a structure that would be affected by incorporation of nucleotides 218 may be used within the scope of the claimed subject matter. For example, a metal wire attached to the surface of or incorporated into a cantilever 116, 212 would be expected to change its resistance as the cantilever 116, 212 bends and the length (and width) of the wire changes. Methods of attaching or incorporating nanowires to cantilevers 116, 212 are known in the art, as are methods of measuring electrical resistance.

Detection Units

A detection unit 118 may be used to detect the deflection and/or resonant frequency of a cantilever 116, 212. The deflection of a cantilever 116, 212 may be detected, for example, using optical and/or piezoresistive detectors 122 (e.g., U.S. Pat. No. 6,079,255) and/or surface stress detectors 122 (e.g. Fritz et al., Science 288[5464]:316-8, 2000).

In an exemplary embodiment of the invention, a piezoresistive resistor may be embedded at the fixed end of the cantilever 116, 212 arm. Deflection of the free end of the cantilever 116, 212 produces stress along the cantilever 116, 212. That stress changes the resistance of the resistor 116, 212 in proportion to the degree of cantilever 116, 212 deflection. A resistance measuring device may be coupled to the piezoresistive resistor to measure its resistance and to generate a signal corresponding to the cantilever 116, 212 deflection. Such piezoresistive detectors 122 may be formed in a constriction at the fixed end of the cantilever 116, 212 such that the detector 122 undergoes even greater stress when the cantilever 116, 212 is deflected (PCT patent application WO97/09584).

Changes in resistance may be used to calculate the change in deflection and/or resonant frequency of the cantilever 116, 212 using methods known in the art. Methods of manufacturing small piezoresistive cantilevers 116, 212 are also known. In a non-limiting example, piezoresistive cantilevers 116, 212 may be formed by defining one or more cantilever 116, 212 shapes on the top layer of a silicon on insulator (SOI) wafer. The cantilever 116, 212 may be doped with boron or another dopant to create a p-type conducting layer. A metal may be deposited for electrical contacts to the doped layer, and the cantilever 116, 212 released by removing the bulk silicon underneath it. Such methods may use known lithography and etching techniques as discussed above.

In some alternative embodiments of the invention, a thin oxide layer may be grown after dopant introduction to reduce the noise inherent in the piezoresistor. Piezoresistor cantilevers 116, 212 may also be grown by vapor phase epitaxy using known techniques. In certain embodiments of the invention, the piezo may be used to drive oscillation of the cantilever 116, 212. By incorporating the piezoresistor into a Wheatstone bridge circuit with reference resistors, the resistivity of the cantilever 116, 212 may be monitored.

In other embodiments of the invention, cantilever 116, 212 deflection and/or resonant frequency may be detected using an optical deflection sensor 118. Such a detection unit 118 comprises a light source 120, e.g. a laser diode or an array of vertical cavity surface emitting lasers (VCSEL), and a position sensitive photodetector 122. A preamplifier may be used to convert the photocurrents into voltages. The light emitted by the light source 120 is directed onto the free end of the cantilever 116, 212 and reflected to one or more photodiodes 122. In certain embodiments of the invention, the free ends of the cantilever 116, 212 may be coated with a highly reflective surface, such as silver, to increase the intensity of the reflected beam 132. Deflection of the cantilever 116, 212 leads to a change in the position of the reflected light beams 132. This change can be detected by the position sensitive photodetector 122 and analyzed to determine the amount of displacement of the cantilever 116, 212. The displacement of the cantilever 116, 212 in turn may be used to determine the additional mass of nucleic acids 214, 220 attached to the cantilever 116, 212. The skilled artisan will realize that the exemplary detection techniques discussed herein may be applied to other types of structures 116, 212, such as a diaphragm or a suspended platform.

In other embodiments of the invention, deflection and/or resonant frequency of the structure 116, 212 may be measured using piezoelectric (PE) and/or piezomagnetic detection units 118 (e.g., Ballato, “Modeling piezoelectric and piezomagnetic devices and structures via equivalent networks,” IEEE Trans. Ultrason. Ferroelectr. Freq. Control 48:1189-240, 2001). Piezoelectric detection units 118 utilize the piezoelectric effects of the sensing element(s) to produce a charge output. A PE detection unit 118 does not require an external power source for operation. The “spring” sensing elements generate a given number of electrons proportional to the amount of applied stress. Many natural and man-made materials, such as crystals, ceramics and a few polymers display this characteristic. These materials have a regular crystalline molecular structure, with a net charge distribution that changes when strained.

Piezoelectric materials may also have a dipole in their unstressed state. In such materials, electrical fields may be generated by deformation from stress, causing a piezoelectric response. Charges are actually not generated, but rather are displaced. When an electric field is generated along the direction of the dipole, mobile electrons are produced that move from one end of the piezoelectric material, through a signal detector 122 to the other end of the piezoelectric material to close the circuit. The quantity of electrons moved is a function of the degree of stress in the piezoelectric material and the capacitance of the system.

The skilled artisan will realize that the detection techniques discussed herein are exemplary only and that any known technique for measuring changes in deflection and/or resonant frequency, or any other mass and/or surface stress dependent properties of a structure 116, 212, may be used.

Nucleic Acids

Nucleic acid molecules 214 to be sequenced may be prepared by any known technique. In one embodiment of the invention, the nucleic acid 214 may be naturally occurring DNA or RNA molecules. Virtually any naturally occurring nucleic acid 214 may be prepared and sequenced by the disclosed methods including, but not limited to, chromosomal, mitochondrial or chloroplast DNA or messenger, heterogeneous nuclear, ribosomal or transfer RNA. Methods for preparing and isolating various forms of nucleic acids 214 are known. (See, e.g., Guide to Molecular Cloning Techniques, eds. Berger and Kimmel, Academic Press, New York, N.Y., 1987; Molecular Cloning: A Laboratory Manual, 2nd Ed., eds. Sambrook, Fritsch and Maniatis, Cold Spring Harbor Press, Cold Spring Harbor, N.Y., 1989). The methods disclosed in the cited references are exemplary only and any variation known in the art may be used.

In cases where single stranded DNA (ssDNA) 214 is to be sequenced, an ssDNA 214 may be prepared from double stranded DNA (dsDNA) by any known method. Such methods may involve heating dsDNA and allowing the strands to separate, or may alternatively involve preparation of ssDNA 214 from dsDNA by known amplification or replication methods, such as cloning into M13. Any such known method may be used to prepare ssDNA or ssRNA 214.

Although the discussion above concerns preparation of naturally occurring nucleic acids 214, virtually any type of nucleic acid 214 that is capable of being attached to a cantilever or equivalent structure 116, 212 could be sequenced by the disclosed methods. For example, nucleic acids 214 prepared by various amplification techniques, such as polymerase chain reaction (PCR™) amplification, could be sequenced. (See U.S. Pat. Nos. 4,683,195, 4,683,202 and 4,800,159.) Nucleic acids 214 to be sequenced may alternatively be cloned in standard vectors, such as plasmids, cosmids, BACs (bacterial artificial chromosomes) or YACs (yeast artificial chromosomes). (See, e.g., Berger and Kimmel, 1987; Sambrook et al., 1989.) Nucleic acid inserts 214 may be isolated from vector DNA, for example, by excision with appropriate restriction endonucleases, followed by agarose gel electrophoresis. Methods for isolation of insert nucleic acids 214 are well known.

Nucleic acids 214 to be sequenced may be isolated from a wide variety of organisms including, but not limited to, viruses, bacteria, pathogenic organisms, eukaryotes, plants, animals, mammals, dogs, cats, sheep, cattle, swine, goats and humans. Also contemplated for use are amplified nucleic acids 214 or amplified portions of nucleic acids 214.

Nucleic acids 214 to be used for sequencing may be amplified by any known method, such as polymerase chain reaction (PCR) amplification, ligase chain reaction amplification, Qbeta Replicase amplification, strand displacement amplification, transcription-based amplification and nucleic acid sequence based amplification (NASBA).

Nucleic Acid Synthesis

Certain embodiments of the invention involve synthesis of complementary DNA 220 using, for example, a DNA polymerase 222. Such polymerases 222 may bind to a primer molecule 224 and add labeled nucleotides 218 to the 3′ end of the primer 224. Non-limiting examples of polymerases 222 of potential use include DNA polymerases 222, RNA polymerases 222, reverse transcriptases 222, and RNA-dependent RNA polymerases 222. The differences between these polymerases 222 in terms of their “proofreading” activity and requirement or lack of requirement for primers 224 and promoter sequences are known in the art. Where RNA polymerases 222 are used, the template molecule 214 to be sequenced may be double-stranded DNA 214. Non-limiting examples of polymerases 222 that may be used include Thermatoga maritima DNA polymerase 222, AmplitaqFS™ DNA polymerase 222, Taquenase™ DNA polymerase 222, ThermoSequenase™ 222, Taq DNA polymerase 222, Qbeta™ replicase 222, T4 DNA polymerase 222, Thermus thermophilus DNA polymerase 222, RNA-dependent RNA polymerase 222 and SP6 RNA polymerase 222.

A number of polymerases 222 are commercially available, including Pwo DNA Polymerase 222 (Boehringer Mannheim Biochemicals, Indianapolis, Ind.); Bst Polymerase 222 (Bio-Rad Laboratories, Hercules, Calif.); IsoTherm™ DNA Polymerase 222 (Epicentre Technologies, Madison, Wis.); Moloney Murine Leukemia Virus Reverse Transcriptase 222, Pfu DNA Polymerase 222, Avian Myeloblastosis Virus Reverse Transcriptase 222, Thermus flavus (Tfl) DNA Polymerase 222 and Thermococcus litoralis (Tli) DNA Polymerase 222 (Promega Corp., Madison, Wis.); RAV2 Reverse Transcriptase 222, HIV-1 Reverse Transcriptase 222, T7 RNA Polymerase 222, T3 RNA Polymerase 222, SP6 RNA Polymerase 222, Thermus aquaticus DNA Polymerase 222, T7 DNA Polymerase 222 +/−3′→5′ exonuclease, Klenow Fragment of DNA Polymerase I 222, Thermus ‘ubiquitous’ DNA Polymerase 222, and DNA polymerase I 222 (Amersham Pharmacia Biotech, Piscataway, N.J.). Any polymerase 222 known in the art capable of template dependent polymerization of labeled nucleotides 218 may be used. (See, e.g., Goodman and Tippin, Nat. Rev. Mol. Cell Biol. 1(2):101-9, 2000; U.S. Pat. No. 6,090,589). Methods of using polymerases 222 to synthesize nucleic acids 220 from labeled nucleotides 218 are known (e.g., U.S. Pat. Nos. 4,962,037; 5,405,747; 6,136,543; 6,210,896).

Primers

Generally, primers 224 are between ten and twenty bases in length, although longer primers 224 may be employed. In certain embodiments of the invention, primers 224 are designed to be exactly complementary in sequence to a known portion of a template nucleic acid 214. Known primer 224 sequences may be used, for example, where primers 224 are selected for identifying sequence variants adjacent to known constant chromosomal sequences, where an unknown nucleic acid 214 sequence is inserted into a vector of known sequence, or where a native nucleic acid 214 has been partially sequenced. Methods for synthesis of primers 224 are known and automated oligonucleotide synthesizers are commercially available (e.g., Applied Biosystems, Foster City, Calif.; Millipore Corp., Bedford, Mass.). Primers 224 may also be purchased from commercial vendors (e.g. Midland Certified Reagents, Midland, Tex.).

Alternative embodiments of the invention may involve sequencing a nucleic acid 214 in the absence of a known primer 224 binding site. In such cases, it may be possible to use random primers 224, such as random hexamers 224 or random oligomers 224 of 7, 8, 9, 10, 11, 12, 13, 14, 15 bases or greater length, to initiate polymerization.

Nucleic Acid Attachment

In various embodiments of the invention, a nucleic acid molecule 214 may be attached to a structure 116, 212 by either non-covalent or covalent binding. In a nonlimiting example, attachment may occur by coating a structure 116, 212 with streptavidin or avidin and then binding of biotinylated nucleic acids 214 and/or primers 224. In different embodiments, the surface of the structure 116, 212 and/or the nucleic acid molecule 214 to be attached may be modified with various known reactive groups to facilitate attachment.

For example, the surface may be modified with aldehyde, carboxyl, amino, epoxy, sulfhydryl, photoactivated or other known groups. Surface modification may utilize any method known in the art, such as coating with silanes that contain reactive groups. Non-limiting examples include aminosilane, azidotrimethylsilane, bromotrimethylsilane, iodotrimethylsilane, chlorodimethylsilane, diacetoxydi-t-butoxysilane, 3-glycidoxypropyltrimethoxysilane (GOP) and aminopropyltrimethoxysilane (APTS). Silanes and other surface coatings for attaching nucleic acids may be obtained from commercial sources (e.g., United Chemical Technologies, Bristol Pa.).

Nucleic acids 214 may also be modified with various reactive groups to facilitate attachment, although in certain embodiments of the invention discussed below, unmodified nucleic acids 214 may also be attached to surfaces. In particular embodiments, nucleic acids 214 may be modified at their 5′ or 3′ ends and/or on internal residues to contain a surface reactive group, such as a sulfhydryl, amino, aldehyde, carboxyl or epoxy group or photoreactive group. In particular embodiments of the invention, nucleic acids 214 may be modified with groups for non-covalent attachment to surfaces, such as biotin, streptavidin, avidin, digoxigenin, fluorescein or cholesterol. Modified nucleic acids, oligonucleotides and/or nucleotides may be obtained from commercial sources (see, e.g. http://www.operon.com/store/desref.php) or may be prepared using any method known in the art.

In particular embodiments of the invention, attachment may take place by direct covalent attachment of 5′-phosphorylated nucleic acids 214 to chemically modified structures 116, 212 (Rasmussen et al., Anal. Biochem. 198:138-142, 1991). The covalent bond between the nucleic acid 214 and the structure 116, 212 may be formed, for example, by condensation with a water-soluble carbodiimide. This method facilitates a predominantly 5′-attachment of the nucleic acids 214 via their 5′-phosphates. In certain embodiments of the invention a template nucleic acid 214 may be immobilized via its 3′ end to allow polymerization of a complementary nucleic acid 220 to proceed in a 5′ to 3′ manner.

Attachment may occur by coating a structure 116, 212 with poly-L-Lys (lysine), followed by covalent attachment of either amino- or sulfhydryl-modified nucleic acids 214 using bifunctional crosslinking reagents (Running et al., BioTechniques 8:276-277, 1990; Newton et al., Nucleic Acids Res. 21:1155-62, 1993). In alternative embodiments of the invention, nucleic acids 214 may be attached to a structure 116, 212 using photopolymers that contain photoreactive species such as nitrenes, carbenes or ketyl radicals (See U.S. Pat. Nos. 5,405,766 and 5,986,076). Attachment may also occur by coating the structure 116, 212 with metals such as gold, followed by covalent attachment of amino- or sulfhydryl-modified nucleic acids 214.

Bifunctional cross-linking reagents may be of use for attachment. Exemplary cross-linking reagents include glutaraldehyde (GAD), bifunctional oxirane (OXR), ethylene glycol diglycidyl ether (EGDE), and carbodiimides, such as 1-ethyl-3-(3-dimethylaminopropyl) carbodiimide (EDC). In some embodiments of the invention, structure 116, 212 functional groups may be covalently attached to cross-linking compounds to reduce steric hindrance between nucleic acid molecules 214 and polymerases 222. Typical cross-linking groups include ethylene glycol oligomers and diamines.

In certain embodiments of the invention a capture oligonucleotide 224 may be bound to a structure 116, 212. The capture oligonucleotide 224 may hybridize with a complementary sequence on a template nucleic acid 214. Once a template nucleic acid 214 is bound, the capture oligonucleotide may be used as a primer 224 for nucleic acid polymerization.

The number of nucleic acids 214 to be attached to each structure 116, 212 will vary, depending on the sensitivity of the structure 116, 212 and the noise level of the system. Large cantilevers 116, 212 of about 500 μm in length may utilize as many as 1010 molecules of attached nucleic acids 214 per cantilever 116, 212. However, using smaller cantilevers 116, 212 the number of attached nucleic acids 214 may be greatly reduced. Determining the number of attached nucleic acids 214 required to generate a usable signal is well within the skill in the art.

Patterning of Nucleic Acids Attached to a Structure

In particular embodiments of the invention, nucleic acids 214 may be attached to the surface of a structure 116, 212 in specific patterns selected to optimize the signal amplitude and decrease background noise. A variety of methods for attaching nucleic acids 214 to surfaces in selected patterns are known in the art and any such method may be used.

For example, thiol-derivatized nucleic acids 214 may be attached to structures 116, 212 that have been coated with a thin layer of gold. The thiol groups react with the gold surface to form covalent bonds (Hansen et al., Anal. Chem. 73:1567-71, 2001). The nucleic acids 214 may be attached in specific patterns by alternative methods. In certain embodiments of the invention, the entire surface of the structure may be coated with gold or an alternative reactive group. Derivatized nucleic acids 214 may be deposited on the surface in any selected pattern, for example by dip-pen nanolithograpy. Alternatively, a gold layer may be etched into selected patterns by known methods, such as reactive-ion beam etching, electron beam or focused ion beam technology. Upon exposure to thiol-modified nucleic acids 214, the nucleic acids 214 will bind to the surface of the structure 116, 212 only where there is a remaining gold layer.

Patterning may also be achieved using photolithographic methods. Photolithographic methods for attaching nucleic acids 214 to surfaces are well known (e.g., U.S. Pat. No. 6,379,895). Photomasks may be used to protect or expose selected areas of a structure 116, 212 to a light beam. The light beam activates the chemistry of a particular area, such as a photoactivable binding group, allowing attachment of template nucleic acids 214 to activated regions and not to protected regions. Photoactivated groups such as azido compounds are known and may be obtained from commercial sources. In certain embodiments of the invention, nano-scale patterns may be deposited on the surface of a structure using known methods, such as dip-pen nanolithograpy, reactive-ion beam etching, chemically assisted ion beam etching, focused ion beam milling, low voltage electron beam or focused ion beam technology or imprinting techniques.

Patterned nucleic acid 214 deposition may be accomplished by any method known in the art. In certain embodiments of the invention, nucleic acid 214 patterns may be deposited using self-assembled monolayers that have been arranged into patterns by known lithographic techniques, such as low voltage electron beam lithograpy. For example, a layer of parylene or equivalent compound could be deposited on the surface of a structure and patterned by liftoff procedures to form a patterned surface for nucleic acid 214 attachment (e.g., U.S. Pat. Nos. 5,612,254; 5,891,804; 6,210,514).

Nucleotide Labels

In certain embodiments of the invention one or more labels may be attached to one or more types of nucleotide 218. A label may consist of a bulky group. Non-limiting examples of labels that could be used include nanoparticles (e.g. gold nanoparticles), polymers, carbon nanotubes, fullerenes, functionalized fullerenes, quantum dots, dendrimers, fluorescent, luminescent, phosphorescent, electron dense or mass spectroscopic labels. Labels of any type may be used, such as organic labels, inorganic labels and/or organic-inorganic hybrid labels. A label may be detected by using a variety of methods, such as a change in resonant frequency of a structure 116, 212, piezoelectric stimulation, structure 116, 212 deflection, and other means of measuring changes in mass and/or surface stress.

Labeled nucleotides 218 may include purine or pyrimidine bases that are linked by spacer arms to labels. Nucleotide 218 bases, sugars and phosphate groups may be modified without compromising hydrogen bond formation or nucleic acid 220 polymerization. Positions of purine or pyrimidine bases that may be modified by addition of labels include, for example, the N2 and N7 positions of guanine, the N6 and N7 positions of adenine, the C5 position of cytosine, thymidine and uracil, and the N4 position of cytosine.

Various labels know in the art that may be used include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, 5-carboxy-2′,4′,5′,7′-tetrachlorofluorescein, 5-carboxyfluorescein, 5-carboxy rhodamine, 6-carboxyrhodamine, 6-carboxytetramethyl amino phthalocyanines, azomethines, cyanines, xanthines, succinylfluoresceins and aminoacridine. These and other labels may be obtained from commercial sources (e.g., Molecular Probes, Eugene, Oreg.). Polycyclic aromatic compounds or carbon nanotubes may also be of use as labels.

Nucleotides 218 that are covalently attached to labels are available from standard commercial sources (e.g., Roche Molecular Biochemicals, Indianapolis, Ind.; Promega Corp., Madison, Wis.; Ambion, Inc., Austin, Tex.; Amersham Pharmacia Biotech, Piscataway, N.J.). Various labels containing reactive groups designed to covalently react with other molecules, such as nucleotides 218, are commercially available (e.g., Molecular Probes, Eugene, Oreg.). Methods for preparing labeled nucleotides 218 are known (e.g., U.S. Pat. Nos. 4,962,037; 5,405,747; 6,136,543; 6,210,896).

Nanoparticles

In Certain embodiments of the invention nanoparticles may be used to label nucleotides 218. In some embodiments of the invention, the nanoparticles are silver or gold nanoparticles. In various embodiments of the invention, nanoparticles of between 1 nm and 100 nm in diameter may be used, although nanoparticles of different dimensions and mass are contemplated. Methods of preparing nanoparticles are known (e.g., U.S. Pat. Nos. 6,054,495; 6,127,120; 6,149,868; Lee and Meisel, J. Phys. Chem. 86:3391-3395, 1982). Nanoparticles may also be obtained from commercial sources (e.g., Nanoprobes Inc., Yaphank, N.Y.; Polysciences, Inc., Warrington, Pa.).

In certain embodiments of the invention, the nanoparticles may be single nanoparticles. Alternatively, nanoparticles may be cross-linked to produce particular aggregates of nanoparticles, such as dimers, trimers, tetramers or other aggregates. In certain embodiments of the invention, aggregates containing a selected number of nanoparticles (dimers, trimers, etc.) may be enriched or purified by known techniques, such as ultracentrifugation in sucrose solutions.

Methods of cross-linking nanoparticles are known (e.g., Feldheim, “Assembly of metal nanoparticle arrays using molecular bridges,” The Electrochemical Society Interface, Fall, 2001, pp. 22-25). Gold nanoparticles may be cross-linked, for example, using bifunctional linker compounds bearing terminal thiol or sulfhydryl groups. Upon reaction with gold nanoparticles, the linker forms nanoparticle dimers that are separated by the length of the linker. In other embodiments of the invention, linkers with three, four or more thiol groups may be used to simultaneously attach to multiple nanoparticles (Feldheim, 2001). The use of an excess of nanoparticles to linker compounds prevents formation of multiple cross-links and nanoparticle precipitation.

In alternative embodiments of the invention, the nanoparticles may be modified to contain various reactive groups before they are attached to linker compounds. Modified nanoparticles are commercially available, such as Nanogold® nanoparticles from Nanoprobes, Inc. (Yaphank, N.Y.). Nanogold® nanoparticles may be obtained with either single or multiple maleimide, amine or other groups attached per nanoparticle. The Nanogold® nanoparticles are also available in either positively or negatively charged form. Such modified nanoparticles may be attached to a variety of known linker compounds to provide dimers, trimers or other aggregates of nanoparticles.

In various embodiments of the invention, the nanoparticles may be covalently attached to nucleotides 218. In alternative embodiments of the invention, the nucleotides 218 may be directly attached to the nanoparticles, or may be attached to linker compounds that are covalently or non-covalently bonded to the nanoparticles. In such embodiments of the invention, rather than cross-linking two or more nanoparticles together the linker compounds may be used to attach a nucleotide 218 to a nanoparticle or a nanoparticle aggregate. In particular embodiments of the invention, the nanoparticles may be coated with derivatized silanes. Such modified silanes may be covalently attached to nucleotides 218 using known methods.

In exemplary embodiments of the invention, the nucleotides 218 may be distinctively labeled with aggregates containing one, two, three or four nanoparticles of similar size. Alternatively, nucleotides 218 may be labeled with individual nanoparticles of different size and mass. Exemplary gold nanoparticles of use are available from Polysciences, Inc. in 5, 10, 15, 20, 40 and 60 nm sizes. In certain embodiments, each different type of nucleotide 218 (A, G, C and T or U) may be labeled with a nanoparticle or nanoparticle aggregate of distinguishable mass.

Information Processing and Control System and Data Analysis

In certain embodiments of the invention, the sequencing apparatus 100 may be interfaced with a data processing and control system 110. In an exemplary embodiment of the invention, the system 110 incorporates a computer 110 comprising a bus or other communication means for communicating information, and a processor or other processing means coupled with the bus for processing information. In one embodiment of the invention, the processor is selected from the Pentium® family of processors, including the Pentium® II family, the Pentium® III family and the Pentium® 4 family of processors available from Intel Corp. (Santa Clara, Calif.). In alternative embodiments of the invention, the processor may be a Celeron®, an Itanium®, a Pentium Xeon® processor or a member of the X-scale® family of processors (Intel Corp., Santa Clara, Calif.). In various other embodiments of the invention, the processor may be based on Intel architecture, such as Intel IA-32 or Intel IA-64 architecture. Alternatively, other processors may be used.

The computer 110 may further comprise a random access memory (RAM) or other dynamic storage device (main memory), coupled to the bus for storing information and instructions to be executed by the processor. Main memory may also be used for storing temporary variables or other intermediate information during execution of instructions by processor. The computer 110 may also comprise a read only memory (ROM) and/or other static storage device coupled to the bus for storing static information and instructions for the processor. Other standard computer 110 components, such as a display device, keyboard, mouse, modem, network card, or other components known in the art may be incorporated into the information processing and control system. The skilled artisan will appreciate that a differently equipped information processing and control system 110 than the examples described herein may be used for certain implementations. Therefore, the configuration of the system 110 may vary.

In particular embodiments of the invention, the detection unit 118 may also be coupled to the bus. A processor may process data from a detection unit 118. The processed and/or raw data may be stored in the main memory. Data on masses for labeled nucleotides 218 and/or the sequence of nucleotide 218 solutions introduced into the analysis chamber 114, 210 may also be stored in main memory or in ROM. The processor may compare the detected changes in mass and/or surface stress to the labeled nucleotide 218 masses to identify the sequence of nucleotides 218 incorporated into a complementary nucleic acid strand 220. The processor may analyze the data from the detection unit 118 to determine the sequence of a template nucleic acid 214.

The information processing and control system 110 may further provide automated control of a sequencing apparatus 100. Instructions from the processor may be transmitted through the bus to various output devices, for example to control pumps, electrophoretic or electro-osmotic leads and other components of the apparatus 100.

It should be noted that, while the processes described herein may be performed under the control of a programmed processor, in alternative embodiments of the invention, the processes may be fully or partially implemented by any programmable or hardcoded logic, such as Field Programmable Gate Arrays (FPGAs), TTL logic, or Application Specific Integrated Circuits (ASICs), for example. Additionally, the methods described may be performed by any combination of programmed general-purpose computer 110 components and/or custom hardware components.

In certain embodiments of the invention, custom designed software packages may be used to analyze the data obtained from the detection unit 118. In alternative embodiments of the invention, data analysis may be performed using a data processing and control system 110 and publicly available software packages. Non-limiting examples of available software for DNA sequence analysis includes the PRISM(tm) DNA Sequencing Analysis Software (Applied Biosystems, Foster City, Calif.), the Sequencher(tm) package (Gene Codes, Ann Arbor, Mich.), and a variety of software packages available through the National Biotechnology Information Facility at website www.nbif.org/links/1.4.1.php.

All of the METHODS and APPARATUS 100 disclosed and claimed herein can be made and executed without undue experimentation in light of the present disclosure. It will be apparent to those of skill in the art that variations may be applied to the METHODS and APPARATUS 100 described herein without departing from the concept, spirit and scope of the claimed subject matter. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the claimed subject matter.

What is claimed is:

1. A method comprising:
a) attaching one or more template nucleic acid molecules to one or more structures;
b) synthesizing one or more complementary nucleic acids from labeled nucleotides;
c) detecting changes in a property of the structures;
d) identifying the incorporated nucleotides from the changes in a property of the structures; and
e) determining the sequence of the template nucleic acid.

2. The method of claim 1, wherein the changes in a property of the structures are a function of the mass of the attached nucleic acids.

3. The method of claim 1, wherein the changes in a property of the structures are a function of the surface stress of the structure.

4. The method of claim 1 wherein the structures are cantilevers.

5. The method of claim 1, wherein the changes in a property of the structures are detected by optical beam detection, piezoelectric detection, piezoresistance detection or electrical resistance detection.

6. The method of claim 1, wherein the changes in property of the structures are detected by changes in the resonant frequency of the structures or in the resistance of an electrical circuit associated with the structure.

7. The method of claim 1, wherein the labeled nucleotides comprise at least one mass labeling group.

8. The method of claim 7, wherein each different type of nucleotide comprises a distinguishable mass labeling group.

9. The method of claim 7, wherein the mass labeling groups are selected from the group consisting of nanoparticles, nanoparticle aggregates, carbon nanotubes, fullerenes, functionalized fullerenes, quantum dots, dendrimers, organic molecules, polymers, heavy atoms, fluorescent labels, luminescent labels and mass spectroscopic labels.

10. The method of claim 1, further comprising hybridizing primers to the template nucleic acids.

11. The method of claim 10, wherein the labeled nucleotides are covalently attached to the 3′ end of the primer by a polymerase.

12. The method of claim 1, wherein the template nucleic acid molecules are arranged on part of the surface of the structures in a selected pattern.

13. The method of claim 1, wherein only a single type of nucleotide is exposed to the template and complementary nucleic acids at one time.

14. The method of claim 8, wherein four different types of nucleotides are exposed to the template and complementary nucleic acids at the same time.

15. A method for nucleic acid analysis comprising:
a) attaching at least one template nucleic acid to one or more structures;
b) synthesizing at least one complementary nucleic acid segment comprising a selected number of labeled nucleotides;
c) detecting changes in a property of the structures upon incorporation of the labeled nucleotides; and
d) determining the sequence of the nucleic acid segment from the changes in the property of the structures.

16. The method of claim 15, further comprising:
e) replacing the labeled nucleotides in the complementary nucleic acid segment with unlabeled nucleotides;
f) synthesizing an adjacent complementary nucleic acid segment comprising a selected number of labeled nucleotides;
g) detecting changes in a property of the structures upon incorporation of the labeled nucleotides; and
h) determining the sequence of the adjacent complementary nucleic acid segment.

17. The method of claim 16, further comprising repeating (e) through (h) until a nucleic acid sequence is obtained.

18. The method of claim 16, wherein the labeled nucleotides are replaced with unlabeled nucleotides by removing the labels from the labeled nucleotides.

19. The method of claim 15, wherein the structures are cantilevers.

20. The method of claim 19, wherein the property of the structures is a deflection of the cantilevers, a resonant frequency of the cantilevers or the resistance of an electrical circuit associated with the cantilevers.

21. The method of claim 20, wherein a deflection of the cantilevers, a shift in the resonant frequency of the cantilevers or a change in the resistance of an electrical circuit associated with the cantilevers is a function of the mass of the labeled nucleotides.

22. The method of claim 20, wherein a deflection of the cantilevers, a shift in the resonant frequency of the cantilevers or a change in the resistance of an electrical circuit associated with the cantilevers is a function of the surface stress of the cantilever.

23. The method of claim 15, wherein the template nucleic acids are arranged on part of the surface of the structures in a selected pattern.

24. An apparatus comprising:
a) an analysis chamber containing one or more structures;
b) one or more reagent reservoirs in fluid communication with the analysis chamber;
c) a detection unit operably coupled to the structures; and
d) a data processing and control unit.

25. The apparatus of claim 24, further comprising one or more nucleic acids attached to the structures.

26. The apparatus of claim 25, further comprising one or more polymerases in the analysis chamber.

27. The apparatus of claim 24, wherein the structures are cantilevers.

28. The apparatus of claim 24, wherein the detection unit comprises a position sensitive photodetector, a piezoelectric detector or a piezoresistor.

29. The apparatus of claim 24, wherein the detection unit comprises a laser.

30. The apparatus of claim 25, said detection unit to detect changes in mass of nucleic acids attached to said structures and/or the surface stress of said structures.
